(19) United States
(12) Patent Application Publication
(10) Pub. No.: US 2010/0324328 A1
(43) Pub. Date: Dec. 23, 2010

(54) Process For Isolating Mono-Carboxy Substituted Probucol Derivatives

(75) Inventors: Jeremy Stephen Parker, Bristol (GB); Evan William Snape, Bristol (GB)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 12/666,101

(22) PCT Filed: Jun. 25, 2008
(86) PCT No.: PCT/SE08/50765

§ 371 Date: Jun. 29, 2010

Related U.S. Application Data
(60) Provisional application No. 60/946,260, filed on Jun. 26, 2007.

(51) Int. Cl. C07C 67/58

(52) U.S. Cl. 560/191

(57) ABSTRACT

A process for isolating a compound of formula (I)

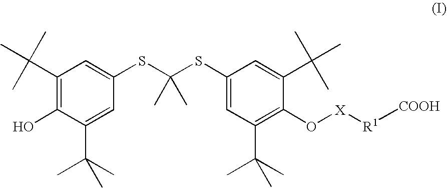

or a salt thereof,
where X and R¹ are as defined in the specification, from a mixture containing it, and the corresponding diacid and dihydroxy derivative,
said process comprising
(i) adding to an organic solution containing said compounds, water and a one or more salts, all of which are bases selected from a carbonate or hydrogen carbonate base,
(ii) separating the aqueous phase containing the compound of formula (II) from the organic phase containing the compounds of formula (I) and (III); then
(iii) recovering the compound of formula (I) from remaining organic phase.

The process provides for efficient isolation of the target compound, even on a large scale.

The present invention relates to a process for isolating chemical compounds in relatively pure form, in particular pharmaceutically active compounds.

Therapeutic compounds for the treatment of for example cardiovascular disease and anti-inflammatory compounds include the compound known as probucol and the mono-substituted derivatives of this including mono-ethers and mono-esters. Probucol has the formula A:

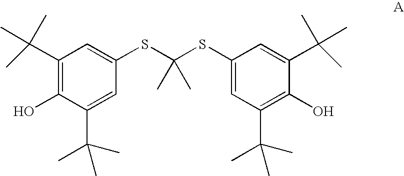

Mono-esters and ethers of probucol, where one of the hydroxyl groups is derivatised are known to be used in the treatment of inflammatory diseases such as rheumatoid arthritis, osteoarthritic, asthma and dermatitis (U.S. Pat. No. 6,147,250), and they have also been reported as being useful in preventing transplant rejection (US Patent Publication No. is 2004/138147).

In particular, mono-esters of probucol such as mono-succinylprobucol (MSP) of formula (B)

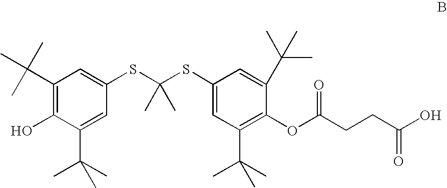

has been shown to be a useful compound in that it may be dosed orally to block VCAM-1 expression, reduce atheroscelerosis and have potent anti-oxidant activity.

Various methods have been described for the preparation of MSP (see for example U.S. Pat. Nos. 5,262,439, 6,147,250 and U.S. Pat. No. 6,323,359 and US Patent Publication Nos. 2004/0204485 and 2005/0267187.

The methods generally require that probucol is used as the starting material, which is esterified. A common by-product of these reactions is the di-succinylprobucol (DSP) of formula C

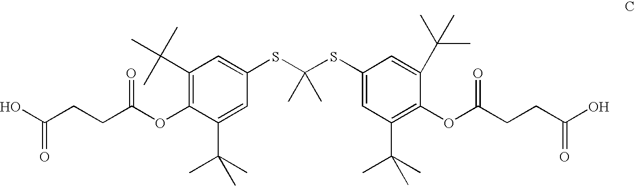

The isolation of the desired MSP from DSP and also from residual probucol is therefore a particular requirement of MSP manufacture.

WO2006/116038 describes a process in which compounds of formula (I)

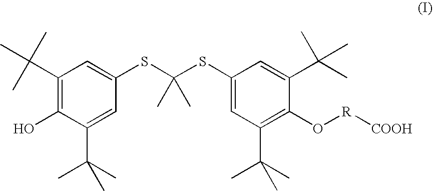

where R is a linker or is selected from —C(O)(CH₂)₂—, —CH₂—, —(CH₂)₂— and —(CH₂)₃—, are is separated from compounds of formula (II)

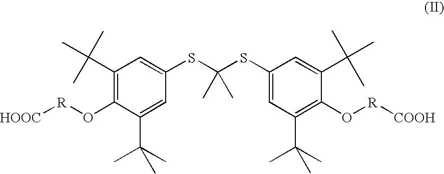

by a procedure which has been described as “partial neutralisation” in which organic solutions of the compounds are either partially acidified or partially basified in order to ensure that at least some of the compound of formula (I) is in the form of a salt, and some is in the form of a free acid. In this state, it is found that the compound of formula (II) is preferentially extracted into an aqueous phase.

This application describes a wide variety of process types which include various combinations of steps selected from operations such as solvent exchange, distillation, crystallisations etc. which lead to the formation of mixtures with differing amounts of the components.

In many of the processes described in this reference, any compound of formula (III)

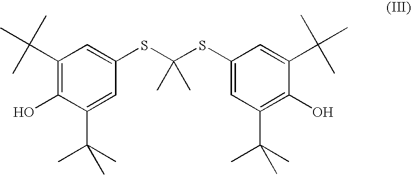

present is removed in an initial crystallisation and isolation step in which a wetcake is comprising a mixture of formula (I) and formula (II) is produced. In procedures where this does not occur, a complex series of solvent exchange steps are required in order to allow the compound of formula (I) to ultimately to be obtained in relatively pure form. In particular, the solvent in which the mixture is initially formed (which is generally tetrahydrofuran), is exchanged in a preliminary step for a non-polar organic solvent such as heptanes.

However, the applicants have found that where the compound of formula (III) is removed by crystallisation in an initial step, the resultant wetcake mixture of formula (I) and formula (II) is difficult to filter and separate due to poor handling properties.

The applicants have found a way to avoid this filtration stage, which does not require the extensive solvent exchange procedures detailed in WO2006/116038.

The present invention provides a process for isolating a compound of formula (I)

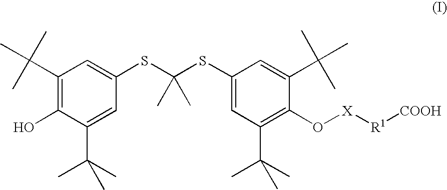

or a salt thereof,
where X is a direct bond, >C(O) or a group >NR² group where R² is hydrogen or a C₁₋₆alkylgroup,
R¹ is a straight or branched C₁₋₁₀ alkylene, straight or branched C₂₋₁₀ alkenylene, straight or branched C₂₋₁₀ alkynylene group, aryl or heterocyclic, any of which may be optionally substituted and wherein any alkylene, alkenylene or alkynylene group may be optionally interposed by an aryl or heterocyclic group;
from a mixture containing it, a compound of formula (II),

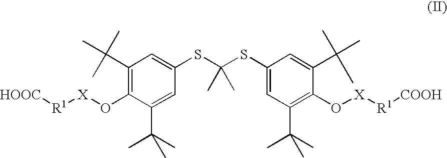

where X and R¹ are as defined in relation to formula (I), and a compound of formula (III)

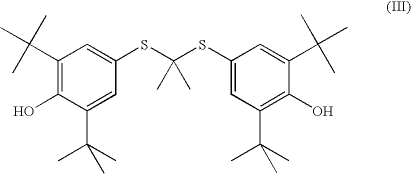

said process comprising
(i) adding to an organic solution containing said compounds, water and one or more salts, all s of which are bases selected from carbonate or hydrogen carbonate bases,
(ii) separating the aqueous phase containing the compound of formula (II) from the organic phase containing the compounds of formula (I) and (III); then
(iii) recovering the compound of formula (I) from remaining organic phase.

The applicants have found that use of carbonate containing bases (which may be organic or inorganic) in the reaction leads to a significant improvement in the separation of the compound of formula (II), as this appears to basify the compound of formula (II) more selectively than the sodium hydroxide used in previous separations. As a result, the compound of formula (II) is more readily extracted into an aqueous phase, so that a substantial portion of the compound of formula (II) is extracted in this single step. As used is herein, the expression “substantial portion” means that the relative proportion of the compound of formula (II) as compared to the total of compounds of formula (I), (II) and (III) is reduced in the organic phase by at least 5% and preferably at least 10%. Since in general, in preparation procedures, the relative percentage of the compound of formula (II) is lower than that of compound (I), such as reduction will lead to a significant purification.

Furthermore, the base does not significantly impact on the pH of the system, so that control of pH is not required during the extraction. The improvements noted are particularly useful when preparing the compound of formula (I) on a large scale.

In step (i) sufficient base is added to fully basify the compound of formula (II), so that the amount of base added is at least the stoichiometric amount needed to convert all carboxyl groups in compound (II) in the mixture into salts. As a result, the compound of formula (II) is extracted into the aqueous phase, from where it may be removed, whilst the compound of formula (I) remains in the organic phase, and is largely in the acid form. Suitable bases include inorganic carbonates or hydrogen carbonates such as alkali and alkaline earth metal carbonates or hydrogen carbonates or mixtures thereof. Particular examples include potassium carbonate, sodium carbonate, sodium hydrogen carbonate, potassium hydrogen carbonate, calcium carbonate or magnesium carbonate. Alternatively the carbonates or hydrogen carbonates may be organic carbonates or such as ammonium carbonates or ammonium hydrogen carbonate. Suitably a single base is added. Thus step (i) above comprises adding to an organic solution containing said compounds, water and a base selected from a carbonate or hydrogen carbonate base.

A particular example of a suitable base for use in step (i) is sodium hydrogen carbonate.

The carbonate or hydrogen carbonate base(s) are added in the absence of other salts in particular chlorides such as sodium chloride as these have the effect of increasing the ionic is strength and retaining compound of formula (II) in the organic phase. [For example as described in Example 79B of WO 2006/116038, a combination of sodium chloride and sodium bicarbonate was used to treat a mixture of compounds (I), (II) and (III) in order to convert the compounds and in particular compound (II) to a sodium salt. However, the relative percentage of compound (II) in the organic phase remained relatively constant throughout this procedure. Even after a subsequent solvent exchange, in which the organic phase was switched to a different organic phase, the wetcake still contained significant amounts of the compound of formula (II).]

In particular, in the compounds of formula (I), (II) and (III), X is a direct bond or a C(O) group. In a particular embodiment, X is a C(O) group.

Suitable optional substituents for R¹ groups include halo, nitro, cyano, haloC₁₋₆alkyl, hydroxyl, carboxyl, acyl, aryl, acyloxy, amino, amido, carboxyl, C₁₋₆alkylamino, di-C₁₋₆alkylamino, arylamino, C₁₋₆ alkoxy, aryloxy, nitro, cyano, sulfonic acid, thiol, imine, sulfonyl, sulfanyl, sulfinyl, sulfamoyl, carbamoyl, C₁₋₆alkoxycarbonyl, phosphonyl, phosphinyl, phosphoryl, phosphine, thioester, thioether, acid halide, oxime, hydrazine, phosphonic acid, phosphonate, C₁₋₁₀cycloalkyl, heterocyclyl or heterocyclyloxy group, wherein any reactive group is optionally protected as necessary. Where R¹ is or contains an aryl or heterocyclic, group, this may also be optionally be substituted by one or more C₁₋₆alkyl, C₂₋₆alkenyl, C₂₋₆alkynyl, heterocyclic or carbocyclic groups, or two adjacent C₁₋₆alkyl, C₂₋₆alkenyl or C₂₋₆alkynyl groups may be joined together to form a fused ring.

In particular R¹ is a straight or branched C₁₋₁₀ alkylene, straight or branched C₂₋₁₀ alkenylene, straight or branched C₂₋₁₀ alkynylene group.

For example, R¹ is a straight or branched C₁₋₆ alkylene group, and in particular is a straight chain C₁₋₆alkylene group such as methylene, ethylene or n-propylene.

As used herein, the expression “aryl” refers to aromatic carbocyclic ring systems such as phenyl or naphthyl. The term “heterocyclic” refers to rings containing up to 20 atoms, at least one of which is a heteroatom selected from oxygen, sulphur or nitrogen. Heterocyclic rings may be mono-, bi- or tricyclic and may be aromatic or non aromatic. Typical examples of heterocyclic rings include pyrrolidinyl, tetrahydrofuryl, tetrahydrofuranyl, pyranyl, purinyl, tetrahydropyranyl, piperazinyl, piperidinyl, morpholino, thiomorpholino, tetrahydropyranyl, imidazolyl, pyrolinyl, pyrazolinyl, indolinyl, dioxolanyl, or 1,4-dioxanyl, aziridinyl, furyl, furanyl, pyridyl, pyridinyl, pyridazinyl, pyrimidinyl, benzoxazolyl, 1,2,4-oxadiazolyl, 1,3,4-oxadiazolyl, 1,3,4-thiadiazole, indazolyl, triazinayl, 1,3,5-triazinyl, thienyl, isothiazolyl, imidazolyl, tetrazolyl, pyrazinyl, benzofuranyl, quinolyl, isoquinolyl, benzothienyl, isobenzofuryl, pyrazolyl, indolyl, isoindolyl, benzimidazolyl, purinyl, carbazolyl, oxazolyl, thiazolyl, benzothiazolyl, isothiazolyl, 1,2,4-thiadiazolyl, isooxazolyl, 1,2,4-oxadiazolyl, 1,3,4-oxadiazolyl, pyrrolyl, quinazolinyl, quinoxalinyl, benzoxazolyl, quinolinyl, isoquinolinyl, cinnolinyl, phthalazinyl, xanthinyl, hypoxanthinyl, pyrazole, imidazole, 1,2,3-triazole, 1,2,4-triazole, 1,2,3-oxadiazole, thiazine, pyridazine, triazolopyridinyl or pteridinyl, morpholine, piperidine, piperazine, pyrrolidine, azetidine, and tetrahydrofuran.

Suitably the organic solution used in step (i) is the solution in which the compound of formula (III) has been reacted to form the compound of formula (I), and which therefore includes some unreacted compound of formula (III), as well as the bi-product of formula (II). Particular solvents used in this way, include, for example, THF.

Suitably some non-polar organic solvent, such as heptane, hexane, toluene, decane, benzene, xylene, mixed heptanes, mesitylene, naphthalene, pentane, octane, nonane, decane, undecane, dodecane, tridecane, tetradecane, pentadecane, hexadecane, heptadecane, eicosane, cyclohexane, or petroleum ether, and mixtures thereof are added to a THF solution in order to avoid problems with phase separations which may occur when THF is used as the organic phase. However, the applicants have found that there is no need to carry out a complete solvent exchange at this stage, and that the presence of residual production solvents such as tetrahydrofuran does not significantly inhibit the isolation procedure. This shortens the reaction time, making the procedure more streamlined for large scale production.

A particularly preferred base for use in step (i) are the alkali metal hydrogen carbonate such as sodium hydrogen carbonate or potassium hydrogen carbonate, and particularly sodium hydrogen carbonate.

Under these conditions, the applicants have found that at least a major portion of the compound of formula (II) is extracted into the aqueous phase, from where it can be removed during step (ii) above.

If required, an additional extraction step may be effected either before or after step (ii), and preferably after step (ii), but certainly before step (iii) in order to eliminate yet more of the compound of formula (II) from the mixture. This is suitably a base extraction process. This may be achieved for example by adding sodium hydroxide together with water and also suitably a polar organic solvent such as those discussed below, so that the resultant salt of the is compound of formula (II) is formed, which is preferentially extracted into the aqueous phase. The amount of sodium hydroxide solution added is suitably sufficient to ensure that at least the compound of formula (II) takes the form of a salt. It is possible that some of the compound of formula (I) may remain in acid form, although this also may be converted to the sodium salt at this stage.

Preferably the polar organic solvent is an organic water soluble solvent. Particular examples include acetone, ethyl acetate, tetrahydrofuran, ethyl acetate, isopropyl acetate, methyl alcohol, ethyl alcohol, isopropyl alcohol, acetonitrile, dimethylformamide, 2-butanone, and mixtures thereof. A particularly preferred solvent in this instance is acetone. Suitably the polar organic solvent is one in which the compound of formula (I) is highly soluble which helps to ensure that the compound of formula (I) remains in the organic phase.

Separation of the aqueous and organic phases following the addition means that further compound of formula (II) is taken out of the reaction mixture prior to step (iii).

Step (iii) may be effected in a variety of ways, which may vary depending upon factors such as the precise nature of the compounds of formula (I) and (III), the purity requirements and the amount of time and resource available to achieve this.

In one embodiment, step (iii) comprises treating the residual organic phase in such a way that the compound of formula (I) or a salt thereof, precipitates out and the compound of formula (III) remains in solution. For example, some of the remaining organic solvent, in particular any polar solvent present may be distilled off until the compound of formula (I) or a salt thereof precipitates out, leaving the compound of formula (III) in solution. Where for example the solution has undergone an additional extraction step that involved the addition of the polar solvent such as acetone or ethyl acetate, removal of the polar solvent in a short distillation step may mean that the compound of formula (I) or a salt thereof crystallises out, leaving the compound of formula (III) in the mother liquor. If necessary or desirable, a non-polar solvent such as heptane may be added at this stage to encourage crystallisation of the compound of formula (I). Recovery of the solid, for example by filtration will lead to the isolation of the compound of formula (I) or a salt in solid form.

In an alternative embodiment, the compound of formula (I) is extracted out of the organic phase remaining at the end of step (ii) into an aqueous phase, leaving the compound of formula (III) in the organic phase. Thereafter, the compound of formula (I) or a salt thereof, may be recovered by re-extracting the compound of formula (I) or the salt back into a is fresh organic phase and precipitating or crystallising it out of the organic phase.

Suitably the fresh organic phase comprises organic solvents as described above, and in particular a combination of both a non-polar and polar organic solvent.

Preferably the transfer back to an organic phase is carried out after acidification where necessary, so that any salt of the compound of formula (I) is in the form of the free acid.

Preferably, in all embodiments, the compound of formula (I) is recovered in the form of an acid. This may be achieved by, where necessary, acidifying the solution at a convenient stage before the crystallisation or precipitation of the product from the organic phase occurs.

Alternatively, the product is obtained in the form of a salt, such as an alkali metal salt, for instance a sodium salt. Salts may provide some handling advantages at this stage, and the sodium salt of MSP has been found to be less prone to static than the corresponding free acid product. In addition, allowing a salt to crystallise may result in a more effective separation from the compound of formula (III).

Salts obtained in this way are suitably changed into the corresponding acids by conventional methods. In particular, they may be dissolved in a suitable solvent such as any of those listed above, in particular a mixed solvent comprising a non-polar solvent such as heptane, and a polar co-solvent such as acetone or ethyl acetate. The solution can then be acidified by the addition of an acid such as hydrochloric acid. The free acid may thereafter be obtained by precipitation, which may be encouraged by distillation of at least some of the polar co-solvent or by seeding or any other conventional method.

If required or necessary, the solids obtained in this way may be subject to further purification by recrystallisation. In the case of the acid form of MSP for instance, this may suitably be achieved by dissolving the product into an organic solvent, in particular a mixture of non-polar and polar organic solvents such as a mixture of heptane and acetone, and distilling off the solvent so that at least the polar solvent such as acetone is removed.

The following examples illustrate the invention.

### EXAMPLE 1

### Isolation of MSP (Acid Form)

A solution of probucol (40 g) in tetrahydrofuran (THF) (62 ml) was formed in a 1L reactor, purged with nitrogen. A solution of benzylmagnesium chloride (23.8 g) in THF (131 ml) was added dropwise over 25 minutes. After a THF line wash (2 ml), the temperature of the mixture was raised to 50° C. and a solution of succinic anhydride (8.3 g) in THF (100 ml) is was added slowly. The mixture was stirred for 15 minutes and then sampled by HPLC, which showed that the mixture had a composition of DSP 12.6%, MSP 62.1% and probucol 22.5%.

Water (27 ml) was added followed by the dropwise addition of concentrated HCl (20 ml) and the mixture stirred for 10 minutes. The mixture was cooled to 20° C. and allowed to stand. The lower aqueous phase was then run off. The reaction mixture was then washed twice with water (68 ml) and brine (12 ml).

Heptane (240 ml) was then added to the reaction mixture with stirring, followed by water (120 ml). With vigorous stirring, a 10% sodium bicarbonate solution (68 ml) was added, the mixture stirred for a further 25 minutes before being allowed to settle. The lower phase was run off, and the composition of the upper organic phase checked by HPLC and found to be DSP (2.01%), MSP (67.1%) and probucol (30.8%).

The reaction mixture was stirred and heated to 50° C. when acetone (105 ml) was added, followed by water (105 ml) and 1.0M sodium hydroxide (6.2 ml). After vigorous stirring for 10 minutes, the mixture was allowed to stand before the lower phase was run off. In this particular example the NaOH extraction was repeated to minimise the DSP level in the organic phase.

The reaction mixture was stirred and cooled to 20° C. and 1.0M HCl (20 ml) was added with stirring for 10 minutes. After being allowed to stand until the layers had settled, the lower aqueous phase was run off.

The residual organic phase was then washed with water (20 ml), and heated so that 290 ml of distillate (219.2 g) were removed, which included acetone and residual THF. The remaining solution was then cooled to 90° C. and heptane (200 ml) added slowly maintaining the temperature at >80° C. After further cooling to 60° C. and seeding with MSP, crystallisation began. Once the mixture had been cooled to 20° C. and left, the solid product was filtered off through a sintered glass funnel and the filtrate recycled for recovery of probucol. This was then washed twice with heptane (70 ml), dried in a vacuum oven to constant weight to yield a solid product (25.1 g, 62.6%) with a composition of 98.3% MSP, 0.04% DSP and 1.7% probucol.

Recrystallisation of the solid (20 g) from a mixture of heptane (150 ml) and acetone (50 ml) by distilling off the acetone yielded MSP with less than 0.25% probucol.

### EXAMPLE 2

### Alternative Preparation of MSP Acid Form

A reaction mixture (60 ml) comprising 10.8% DSP : 58.5% MSP : 28.8% probucol as assessed by HPLC was placed in a reaction vessel and heptane (409.3 mmoles; 60.0 mL; 41.0 g) added with stirring. Water (1.7 moles; 30.0 mL) and sodium hydrogen carbonate solution (14.4 mmoles; 12.0 mL; 13.2 g) were charged to the reactor and stirred at 350 rpm for 30minutes before allowing to stand. The lower phase (53 ml) was then discarded.

The upper phase (106 ml) was then subject to a base extraction by being stirred and acetone (353.7 mmoles; 26.0 mL; 20.5 g) and water (1.4 moles; 26.0 mL; 26.0 g) added. The mixture was stirred and heated to 50° C. Sodium hydroxide solution (1.7 mmoles; 1.7 mL; 1.8 g;) was added and stirred at 350 rpm for 10 minutes before being allowed to stand, whereupon the lower phase (41 ml) was discarded.

Two further acetone washes were carried out, the first by adding acetone (176.8 mmoles; 13.0 mL; 10.3 g;), water (1.1 moles; 20.0 mL; 20.0 g) and sodium hydroxide solution (300.0 μmoles; 300.0 μL; 312.0 mg;) stirring at 350 rpm for 10 minutes before being allowed to stand, whereupon, the lower phase (31 ml) was discarded. To the remaining upper phase was added acetone (353.7 mmoles; 26.0 mL; 20.5 g) and water (1.4 moles; 26.0 mL; 26.0 g). Sodium hydroxide solution (10.0 mmoles; 10.0 mL; 10.4 g) was then added. The reaction mixture was stirred at 50° C. for 10 minutes and then allowed to stand. In this instance, after checking the content on HPLC, it appeared that, in the presence of acetone and under the high pH conditions achieved by using sodium hydroxide as the base, the sodium salt s of MSP was extracted into the aqueous phase. As a result, the upper phase was discarded and the lower aqueous phase returned to the reactor. The aqueous phase was then washed with heptane (136.4 mmoles; 20.0 mL; 13.7 g).

Heptane (409.3 mmoles; 60.0 mL; 41.0 g;) was added to the vessel and the mixture stirred at 50° C. After this, hydrogen chloride (10.0 mmoles; 10.0 mL; 10.1 g) was added and the mixture stirred for a further 10 minutes. On acidification, the MSP (in the form of the free acid was found to be extracted into the organic (heptane) phase. Once allowed to separate, the lower phase (vol=58 ml, pH5) was run off and discarded.

The organic phase was heated to reflux and 30 ml of distillate collected. Mixed heptane (204.7 mmoles; 30.0 mL; 20.5 g) was added and the stirred mixture cooled to 60° C.

A solid product crystallised after 20 minutes. This was cooled to 20° C. and the product filtered off through a small glass sintered funnel. The mother liquor was recycled through the reactor to facilitate removal of some residual product from the walls of the vessel. The solid product was de-liquored and then washed with heptanes (2×15 ml) via the reactor. The solid was pulled free of liquor on the sinter and then dried in a vacuum oven at 50° C. and found to comprise 0.04% DSP : 99.9% MSP : 0.00% Probucol.

### EXAMPLE 3

### Isolation of MSP Sodium Salt

A 1 L reactor was purged with nitrogen and charged with probucol (77.39 mmoles; 40.00 g;) followed by tetrahydrofuran (62.00 mL]) and the solution was stirred (250 rpm) at 25° C. Benzylmagnesium chloride (158.00 mmoles; 81.45 g) was transferred to a dropping funnel under nitrogen and added dropwise at a rate that kept the temperature below 55° C. A tetrahydrofuran (221.20 mmoles; 18.00 ml) line wash was carried out.

Succinic anhydride (82.11 mmoles; 8.30 g) was dissolved in tetrahydrofuran (1.23 moles; 100.00 mL) by stirring in a stopped flask. The reaction mixture was heated to 50° C. and the succinic anhydride solution was added dropwise over 20 minutes. The reaction mixture was stirred for 15 minutes at 50° C.

The agitator speed was increased to 350 rpm and water (1.50 moles; 27.00 mL) and then 32% hydrogen chloride (208.88 mmoles; 20.00 mL) added dropwise. The mixture was stirred and cooled to 20° C. and then allowed to stand. The lower phase was run off (53 ml).

Two water and brine washes were conducted, each by adding water (3.77 moles; 68.00 mL) followed by brine (41.07 mmoles; 12.00 mL), stirring the mixture for 10 minutes before allowing it to stand (5 minutes) and running off the lower aqueous phase.

The remaining organic phase was stirred and heptane (1.64 moles; 240.00 mL) added followed by water (6.66 moles; 120.00 mL). The mixture was stirred at 400 rpm and sodium bicarbonate (81.60 mmoles; 68.00 mL; 74.80 g) added. The mixture was then stirred for 35 minutes before being allowed to stand. The content of both phases was checked on HPLC at this stage, and this revealed that the upper phase (420 ml—clear solution) contained 1.64% DSP : 65.8% MSP : 32.5% probucol, whereas the lower phase (240 ml—yellow solution) contained 62.6% DSP : 22.1% MSP : 0.6% probucol.

The reaction mixture was stirred and heated to 50° C., whereupon acetone (1.43 moles; is 105.00 mL; 82.96 g) and water (5.83 moles; 105.00 mL; 105.00 g) were added to the reactor, which was reheated to 50° C. Then sodium hydroxide (6.40 mmoles; 6.40 mL; 6.66 g;) was added and the mixture stirred at 400 rpm for 10 minutes before being allowed to stand for 10 minutes. The lower phase was then run off.

Acetone (652.97 mmoles; 48.00 mL; 37.92 g) and water (4.44 moles; 80.00 mL; 80.00 g;) were charged to the reactor and allowed to heat to 50° C. Sodium hydroxide (1.20 mmoles; 1.20 mL; 1.25 g) was then added and the mixture stirred at 400 rpm for 10 minutes before allowing to stand for 10 minutes. Again, the lower phase was run off. The residual upper phase was found to contain 0.07% DSP : 66.4% MSP (in the form of the sodium salt): 33.5% probucol.

The organic phase was stirred and water (13.2 moles; 240 ml) added followed by sodium bicarbonate solution (57.60 moles; 48.00 ml; 52.80 g). The mixture was stirred for 60 minutes, whereupon the lower aqueous phase was run off (355 ml) and discarded.

Water (1.11 moles; 20.00 ml) and brine (68.44 mmoles;20.00 ml) were added and the mixture stirred for 5 minutes before allowing to stand. The lower phase was checked on HPLC and found to contain almost no product.

The organic phase was then washed with brine (68.44 mmoles; 20.00 mL; 20.00 g), and then heated to distill off the acetone. The heptane solution containing MSP sodium salt and probucol was then distilled at atmospheric pressure collecting 160 ml of distillate.

The solution was cooled to 90° C. and heptane (1.09 moles; 160.00 mL; 109.38 g;) added slowly maintaining the temperature above 80° C. The solution was cooled to 60° C., and seeded with (10 mg) MSP sodium salt. The solution was then allowed to self cool and stir over the weekend at room temp.

The resultant solid was filtered off using a glass sintered funnel. The product was de-liquored and the liquors returned to the reactor and stirred vigorously to facilitate removal of product residues in the reactor.

The liquors were filtered through the sinter and the product de-liquored. Heptane (409.32 mmoles; 60.00 mL; 41.02 g;) was charged to the reactor and stirred for 5 minutes in the reactor before using to wash the product. This was repeated with a further charge of heptane (409.32 mmoles; 60.00 mL; 41.02 g). is The product was thoroughly de-liquored and then after pulling dry on the sinter for 20 minutes, was dried in a vacuum oven at 40° C. to constant weight. Wt of product=25.8 g. which was determined by HPLC to comprise: 0.00% DSP : 98.7% MSP (sodium salt): 1.28% probucol.

1. A process for isolating a compound of formula (I) 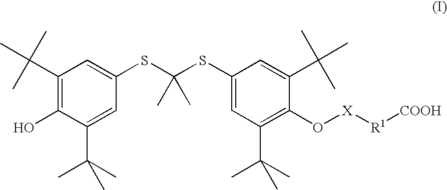
or a salt thereof,
where X is a direct bond, >C(O) or a group >NR² group where R² is hydrogen or a C₁₋₆alkyl group,
R¹ is a straight or branched C₁₋₁₀ alkylene, straight or branched C₂₋₁₀ alkenylene, straight or branched C₂₋₁₀ alkynylene group, aryl or heterocyclic, any of which may be optionally substituted and wherein any alkylene, alkenylene or alkynylene group may be optionally interposed by an aryl or heterocyclic group,
from a mixture containing it, a compound of formula (II), 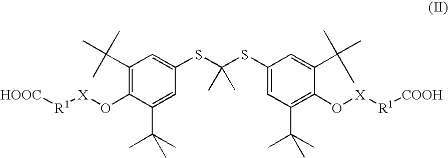
where X and R¹ are as defined in relation to formula (I),
and a compound of formula (III) 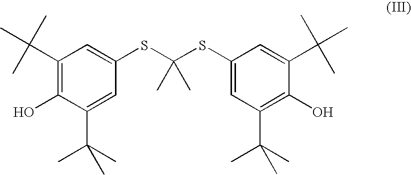
said process comprising
(i) adding to an organic solution containing said compounds, water and one or more salts, all of which are bases selected from carbonate or hydrogen carbonate bases,
(ii) separating the aqueous phase containing the compound of formula (II) from the organic phase containing the compounds of formula (I) and (III); then
(iii) recovering the compound of formula (I) from remaining organic phase.

2. A process according to claim 1 wherein X is a C(O) group.

3. A process according to claim 1 wherein R¹ is a straight or branched C₁₋₁₀ alkylene, straight or branched C₂₋₁₀ alkenylene, straight or branched C₂₋₁₀ alkynylene group.

4. A process according to claim 3 wherein R¹ is methylene, ethylene or n-propylene.

5. A process according to claim 1 wherein the organic solution used in step (i) is the solution in which the compound of formula (III) has been reacted to form the compound of formula (I).

6. A process according to claim 5 wherein the organic solution comprises tetrahydrofuran.

7. A process according to claim 1 wherein the organic solution used in step (i) comprises a non-polar organic solvent.

8. A process according to claim 7 wherein said non-polar organic solvent is heptane, hexane, toluene, decane, benzene, xylene or mixed heptanes.

9. A process according to claim 1 wherein a single base salt is added in step (i).

10. A process according to claim 1 wherein the base used in step (i) is an alkali or alkaline earth metal carbonate or hydrogen carbonate.

11. A process according to claim 10 wherein the base is sodium hydrogen carbonate.

12. A process according to claim 1 wherein at least one additional extraction step in which compound of formula (II) is eliminated from the mixture is effected either before or after step (ii) and before step (iii).

13. A process according to claim 12 wherein the additional extraction step comprises adding sodium hydroxide together with water, so that the sodium salt of the compound of formula (II) is formed, which is preferentially extracted into the aqueous phase.

14. A process according to claim 13 wherein a polar organic solvent is added.

15. A process according to claim 1 wherein step (iii) comprises treating the residual organic phase in such a way that the compound of formula (I) or a salt thereof, precipitates out and the compound of formula (III) remains in solution.

16. A process according to claim 15 wherein the organic phase comprises a polar co-solvent in the organic phase is removed by distillation to cause the compound of formula (I) to precipitate out.

17. A process according to claim 15 wherein a non-polar solvent is added to encourage crystallisation of the compound of formula (I).

18. A process according to claim 1 wherein in step (iii), the compound of formula (I) is extracted out of the organic phase remaining at the end of step (ii) into an aqueous phase, leaving the compound of formula (III) in the organic phase, re-extracting the compound of formula (I) or a salt thereof into a fresh organic phase, and recovering the compound of formula (I) or a salt thereof from the said organic phase.

19. A process according to claim 1 wherein the compound of formula (I) is recovered in the form of an acid.

20. A process according to claim 1 wherein the product is obtained in the form of an alkali metal salt, and the salt obtained is subsequently converted into the acid form.

21. A process according to claim 1 wherein the product obtained is recrystallised.
